(19) United States
(12) Patent Application Publication
(10) Pub. No.: US 2021/0196593 A1
(43) Pub. Date: Jul. 1, 2021

(54) POLYACRYLATE OIL GEL COMPOSITION

(71) Applicants: DOW GLOBAL TECHNOLOGIES LLC, Midland, MI (US); ROHM AND HAAS COMPANY, Collegeville, PA (US)

(72) Inventors: Liang CHEN, Midland, MI (US); Yunshen CHEN, Royersford, PA (US); Xiang Qian LIU, Norristown, PA (US); Michael T. PETR, Collegeville, PA (US); Jodi A. THOMAS, Midland, MI (US); Fanwen ZENG, Audubon, PA (US)

(73) Assignees: Dow Global Technologies LLC, Midland, MI (US); Rohm and Haas Company, Collegeville, PA (US)

(21) Appl. No.: 15/777,367

(22) PCT Filed: Dec. 7, 2016
(86) PCT No.: PCT/US2016/065339

§ 371 Date: May 18, 2018

Related U.S. Application Data
(60) Provisional application No. 62/266,960, filed on Dec. 14, 2015.

(51) Int. Cl. A61K 8/37; A61K 8/81; A61Q 19/00; C08L 33/10; C08L 91/00; A61K 8/04

(52) U.S. Cl. CPC A61K 8/375; A61K 8/8152; A61Q 19/00; A61K 2800/412; C08L 91/00; A61K 8/042; A61K 2800/654; C08L 33/10

(57) ABSTRACT

Provided are personal care compositions comprising a polyacrylate oil gel composition comprising (a) hydrophobic ester oil, and (b) one or more polymers comprising polymerized units derived from (i) 85 to 100 weight % of C₄-C₈ (meth)acrylate monomers, and (ii) 0 to 15 weight % of high T_{g} monoethylenically unsaturated monomers having a T_{g} of more than 90° C. after polymer formation.

### FIELD OF THE INVENTION

This invention relates generally to polyacrylate oil gels that are useful in personal care formulations. The polyacrylate oil gels contain hydrophobic oil ester and acrylic copolymers.

### BACKGROUND

Personal care compositions contain a variety of additives that provide a wide array of benefits to the composition. One class of additives are oil thickeners that provide viscosity enhancements and impart good aesthetics, such as good sensory feel and clarity. One type of oil thickening agent known in the art are cellulose-based polymers and polyamides. These thickeners, however, come with certain drawbacks, including insufficient viscosity enhancement, high formulation temperature, and lack of consistency in viscosity control in consumer product formulations.

To this end, polyacrylate oil gels have been utilized in the art. For example, WO 2014/204937 A1 discloses personal care compositions comprising a polyacrylate oil gel containing a cosmetically acceptable hydrophobic ester oil and a polymer including at least two polymerized units. The prior art does not, however, disclose a polyacrylate oil gel according to the present invention which achieves the significant viscosity performance at low formulation temperatures while also providing a clear formulation.

Accordingly, there is a need to develop thickeners that provide significant viscosity enhancements, while not suffering from the drawbacks of the prior art.

### STATEMENT OF INVENTION

One aspect of the invention provides a polyacrylate oil gel composition comprising (a) hydrophobic ester oil, and (b) one or more polymers comprising polymerized units derived from (i) 85 to 100 weight % of C₄-C₈ (meth)acrylate monomers, and (ii) 0 to 15 weight % of high T_{g} monoethylenically unsaturated monomers having a T_{g} of more than 90° C. after polymer formation.

In another aspect, the invention provides a personal care composition comprising a polyacrylate oil gel comprising (a) one or more aliphatic C₈-C₂₄ alkyl triglycerides, (b) one or more polymers comprising polymerized units derived from (i) 80 to 90 weight % of butyl methacrylate, and (ii) 10 to 20 weight % of ethylhexyl methacrylate, and (c) a dermatologically acceptable carrier.

### BRIEF DESCRIPTION OF THE FIGURES

FIG. 1 shows the rheology profile of a polyacrylate oil gel composition in accordance with the present invention as compared as against a non-inventive sample.

### DETAILED DESCRIPTION

The inventors have now surprisingly found that polyacrylate oil gel compositions comprising hydrophobic ester oil and polymers having a high weight percent of polymerized units derived from C₄-C₈ (meth)acrylate provide significant viscosity enhancements while retaining clarity in personal care formulations. Accordingly, the present invention provides in one aspect a polyacrylate oil gel composition comprising (a) hydrophobic oil ester, and (b) one or more polymers comprising polymerized units derived from (i) 85 to 100 weight % of C₄-C₈ (meth)acrylate monomers, and (ii) 0 to 15 weight % of high T_{g} monoethylenically unsaturated monomers having a T_{g} of more than 90° C. after polymer formation.

In the present invention, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, and topical medicines. In the present invention, “personal care” is also intended to refer to hair care compositions including, for example, shampoos, leave-on conditioners, rinse-off conditioners, styling gels, pomades, hair coloring products (e.g., two-part hair dyes), hairsprays, and mousses. Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylate” refers to either acrylate or methacrylate, and the term “(meth)acrylic” refers to either acrylic or methacrylic. As used herein, the term “substituted” refers to having at least one attached chemical group, for example, alkyl group, alkenyl group, vinyl group, hydroxyl group, carboxylic acid group, other functional groups, and combinations thereof.

As used herein, the terms “glass transition temperature” or “T_{g}” refers to the temperature at or above which a glassy polymer will undergo segmental motion of the polymer chain. Glass transition temperatures of a polymer can be estimated by the Fox equation (Bulletin of the American Physical Society, 1 (3) Page 123 (1956)) as follows:

1/T_{g}=w₁/T_{g(1)}+w₂/T_{g(2)}

For a copolymer, w₁ and w₂ refer to the weight fraction of the two comonomers, and T_{g(1)} and T_{g(2)} refer to the glass transition temperatures of the two corresponding homopolymers made from the monomers. For polymers containing three or more monomers, additional terms are added (wₙ/T_{g(n)}). The T_{(g)} of a polymer can also be calculated by using appropriate values for the glass transition temperatures of homopolymers, which may be found, for example, in “Polymer Handbook,” edited by J. Brandrup and E. H. Immergut, Interscience Publishers. The T_{g} of a polymer can also be measured by various techniques, including, for example, differential scanning calorimetry (“DSC”). When a monomer is said to have a certain T_{g}, it is meant that a homopolymer made from that monomer has that T_{g}.

The inventive personal care compositions include one or more polymers comprising C₄-C₈ (meth)acrylate monomers. Suitable C₄-C₈ (meth)acrylate monomers include, for example, n-butyl (meth)acrylate, i-butyl (meth)acrylate, 2-ethylhexyl (meth)acrylate, cyclohexyl (meth)acrylate, n-octyl (meth)acrylate, phenyl (meth)acrylate, benzyl (meth)acrylate, and 2-phenylethyl (meth)acrylate. Preferably, the C₄-C₈ (meth)acrylate monomers comprise one or more of i-butyl methacrylate, n-butyl methacrylate, and ethylhexyl methacrylate. In certain embodiments, the polymer comprises polymerized units of C₄-C₈ (meth)acrylate monomers in an amount of from 80 to 100 weight %, preferably from 89.5 to 100 weight %, and even more preferably from 99 to 100 weight %, based on the total weight of the polymer. In certain embodiments, the polymer comprises polymerized units derived from 50 to 100 weight % butyl (meth)acrylate monomers, based on the total weight of the polymer.

In certain embodiments, the polymers further comprise high T_{g} monoethylenically unsaturated monomers having a T_{g} of more than 90° C. after polymer formation, as calculated by the Fox equation. Suitable high T_{g} monoethylenically unsaturated monomers include, for example, methyl (meth)acrylate, carboxylic acid containing monomers (e.g., (meth)acrylic acid, itaconic acid, fumaric acid, and maleic acid), styrene, substituted styrene (e.g., chlorostyrene, methylstyrene (e.g., α-methylstyrene), and ethyl styrene). In certain preferred embodiments, high T_{g} monoethylenically unsaturated monomers comprise one or more of methyl methacrylate, t-butyl methacrylate, styrene, and isobornyl methacrylate. Preferably, the high T_{g} monoethylenically unsaturated monomers comprise methyl methacrylate. In certain embodiments, the polymer comprises polymerized units of high T_{g} monoethylenically unsaturated monomers in an amount of from 0.01 to 15 weight %, preferably from 0.1 to 10 weight %, and even more preferably from 1 to 5 weight %, based on the total weight of the polymer.

The polymers can also include crosslinkers, such as a monomer having two or more non-conjugated ethylenically unsaturated groups, i.e., a multiethylenically unsaturated monomer. Suitable multiethylenically unsaturated monomers include, for example, di- or tri-allyl ethers and di- or tri-(meth)acrylyl esters of diols or polyols (e.g., trimethylolpropane diallyl ether, trimethylolpropane triacrylate, ethylene glycol dimethacrylate), di- or tri-allyl esters of di- or tri-acids, (e.g. diallyl phthalate), allyl (meth)acrylate, divinyl sulfone, triallyl phosphate, and divinylaromatics (e.g., divinylbenzene). Preferably, the crosslinkers comprise allyl (meth)acrylate. In certain embodiments, the inventive copolymers comprise polymerized units of crosslinker monomers in an amount of from 0.01 to less than 0.3 weight %, preferably from 0.02 to 0.08 weight %, and more preferably from 0.04 to 0.06 weight %, based on the total weight of the polymer.

In certain embodiments, the polymers have an average particle size of from 50 to 500 nm, preferably of from 100 to 200 nm, and more preferably of from 130 to 140 nm. Polymer molecular weights can be measured by standard methods such as, for example, size exclusion chromatography or intrinsic viscosity. In certain embodiments, the polymers of the present invention have a weight average molecular weight (M_{w}) of 10,000,000 or less, preferably 8,500,000 or less, and more preferably 7,000,000 or less as measured by gel permeation chromatography. In certain embodiments, the copolymer particles have a M_{w} of 50,000 or more, preferably 100,000 or more, and more preferably 200,000 or more, as measured by gel permeation chromatography. In certain embodiments, the polymers are present in the polyacrylate oil gel in an amount of from 0.1 to 20 weight %, preferably from 1 to 13 weight %, and more preferably from 4 to 6 weight %, based on the total weight of the polyacrylate oil gel composition.

Suitable polymerization techniques for preparing the polymers contained in the inventive personal care compositions include, for example, emulsion polymerization and solution polymerization, preferably emulsion polymerization, as disclosed in U.S. Pat. No. 6,710,161. Aqueous emulsion polymerization processes typically are conducted in an aqueous reaction mixture, which contains at least one monomer and various synthesis adjuvants, such as the free radical sources, buffers, and reductants in an aqueous reaction medium. In certain embodiments, a chain transfer agent may be used to limit molecular weight. The aqueous reaction medium is the continuous fluid phase of the aqueous reaction mixture and contains more than 50 weight % water and optionally one or more water miscible solvents, based on the weight of the aqueous reaction medium. Suitable water miscible solvents include, for example, methanol, ethanol, propanol, acetone, ethylene glycol ethyl ethers, propylene glycol propyl ethers, and diacetone alcohol. In certain embodiments, the aqueous reaction medium contains more than 90 weight % water, preferably more than 95 weight % water, and more preferably more than 98 weight % water, based on the weight of the aqueous reaction medium.

The polymers of the present invention may be isolated by a spray drying process. While spray drying is one preferred embodiment of how to produce the dry powder, other suitable methods include, for example, freeze drying, a two-step process including the steps of (i) pan drying the emulsion and then (ii) grinding the pan dried material into a fine powder, coagulation of the acrylic emulsion and collection of the powder by filtration followed by washing and drying, fluid bed drying, roll drying, and freeze drying. Suitable techniques for spray drying the polymer beads of the present invention are known in the art, for example, as described in US 2014/0113992 A1. In certain embodiments, anti-caking agents are used when spray drying the polymer beads. Suitable anti-caking agents include, for example, mineral fillers (e.g., calcium carbonate, kaolin, titanium oxide, talc, hydrated alumina, bentonite, and silica), solid polymer particles with a T_{g} or Tₘ greater than 60° C. (e.g., polymethylmethacrylate, polystyrene, and high density polyethylene), and water soluble polymers with a T_{g} greater than 60° C. (e.g., polyvinyl alcohol and methylcellulose). The anti-caking agent can be mixed in the acrylic suspension prior to spray drying or introduced as a dry powder in the spray drying process. In certain embodiments, the anti-caking agent coats the polymer beads to prevent the beads from sticking to each other inner wall of the dryer. In certain embodiments, the anti-caking agent is present in an amount of from 0 to 20 weight %, and more preferably from 0.01 to 10 weight %, based on the total weight of the polymer beads.

The polyacrylate oil gel compositions of the present invention also contain a cosmetically acceptable hydrophobic ester oil. In general, any hydrophobic ester oil or mixtures thereof which are toxicologically safe for human or animal use may constitute the oil base of the present invention. In certain embodiments, the hydrophobic ester oil comprises aliphatic C₈-C₂₄ alkyl triglycerides. Suitable hydrophobic ester oils include, for example, caprylic/capric triglycerides, saturated fatty esters and diesters (e.g., isopropyl palmitate, octyl palmitate, butyl stearate, isocetyl stearate, octadodecyl stearate, octadodecyl stearoyl stearate, diisopropyl adipate, and dioctyl sebacate), and animal oils and vegetable oils (e.g., mink oil, coconut oil, soybean oil, palm oil, corn oil, cocoa butter, sesame oil, sunflower oil, jojoba oil, olive oil, and lanolin oil). In certain embodiments, the hydrophobic ester oil is diffused in an oil base. Suitable oil bases include any oil or mixture of oils which are conventionally used in personal care products including, for example, paraffin oils, paraffin waxes, and fatty alcohols (e.g., stearyl alcohol, isostearyl alcohol, and isocetyl alcohol). In certain embodiments, the hydrophobic ester oils are present in the polyacrylate oil gel in an amount of from 80 to 99.9 weight %, preferably from 87 to 99 weight %, and more preferably from 94 to 96 weight %, based on the total weight of the polyacrylate oil gel composition.

Polyacrylate oil gels according to the present invention may be formulated by conventional mixing processes known to those skilled in the art. In certain embodiments, the formulation temperature is from 25° C. to 150° C., preferably from 50° C. to 100° C., and more preferably from 60° C. to 80° C. In certain embodiments, the inventive personal care composition includes the polyacrylate oil gel described herein in an amount of at least 0.5 weight %, at least 2 weight %, or at least 4 weight %, by weight of the composition. In certain embodiments, the inventive skin care compositions comprise the particles described herein in an amount of no more than 25 weight %, no more than 30 weight %, or no more than 40 weight %, by weight of the composition.

The inventive personal care compositions also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. The aqueous solutions may contain cosolvents, e.g., water miscible cosolvents. Suitable water miscible cosolvents include, for example, ethanol, propanol, acetone, ethylene glycol ethyl ethers, propylene glycol propyl ethers, and diacetone alcohol. In some embodiments, the composition contains from about 99.99 to about 50 percent by weight of the dermatologically acceptable carrier, based on the total weight of the composition.

Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, vitamins (e.g., Vitamin C) and derivatives thereof, silicones, and fatty alcohols. The amount of option ingredients effective for achieving the desired property provided by such ingredients can be readily determined by one skilled in the art.

Some embodiments of the invention will now be described in detail in the following Examples.

### EXAMPLES

### Example 1

### Preparation of Exemplary Polymer and Comparative Polymers

Exemplary polymers in accordance with the present invention and comparative polymers contain the components recited in Table 1.

TABLE 1
Exemplary Polymer Beads and Comparative Copolymer Particles

| Sample | Monomer (wt %) |
|---|---|
| P1 | 100 nBMA |
| P2 | 90 iBMA/10 MMA |
| P3 | 100 iBMA // 0.04 ALMA |
| P4 | 100 iBMA // 0.06 ALMA |
| P5 | 90 iBMA/10 EHMA // 0.06 ALMA |
| P6 | 80 iBMA/20 EHMA // 0.06 ALMA |
| P7 | 50 iBMA/50 EHMA |
| C1* | 80 nBMA/20 MMA |
| C2* | 100 EHMA // 0.06 ALMA |
| C3* | Stage 1 (70%): 40 EHA/20 BA/38.5 MMA/1.5 MAA // 0.125 |
| | ALMA |
| | Stage 2 (30%): 99 MMA/1 MAA |
| C4* | Styrene-ethylene/butylene-styrene triblock copolymer |
| | (KRATON A1535) |
| C5* | Styrene-ethylene/butylene-styrene triblock copolymer |
| | (KRATON A1536) |

nBMA = n-butyl methacrylate
iBMA = isobutyl methacrylate
MMA = methyl methacrylate
EHMA = ethylhexyl methacrylate
EHA = ethylhexyl acrylate
MAA = methacrylic acid
*Comparative

Synthesis of exemplary polymer P5 was carried out as follows. A 1 liter, 4-neck round bottom flask was equipped with an overhead stirrer, thermocouple, heating mantle, adapter inlet, Claisen head fitted with a water condenser and nitrogen inlet, and an inlet 10 adapter. 230 g deionized water was added to the flask and heated to 75° C. under nitrogen. 1.0 g of aqueous solution containing 0.010 g FeSO₄.H₂O and 0.010 g versene was added to the flask, followed by metering in a monomer emulsion containing 60.0 g deionized water, 0.06 g ALMA, 10.0 g EHMA, 1.785 g SLS (28%), and 90.0 g iBMA over 60 minutes with the addition of 0.10 g tert-butyl hydroperoxide (in 10.0 g deionized water) and 0.10 g isoascorbic acid (in 10.0 g of deionized water) over 60 minutes. The reaction mixture was then held at 75° C. for 10 minutes, after which 0.10 g of tert-butyl hydroperoxide (in 10.0 g deionized water) and 0.10 g of isoascorbic acid (in 10.0 g of deionized water) were concurrently added over 60 minutes while the temperature of the reaction mixture was cooled to 60° C. The mixture was then kept at 60° C. for another 10 minutes. The final latex was cooled to room temperature and filtered.

Exemplary polymers P2-P4, P6, and P7, and comparative polymers C2 were prepared substantially as described above, with the appropriate changes in monomer amounts as recited in Table 1.

Synthesis of exemplary polymer P1 and comparative polymer C1 was carried out using a standard emulsion polymerization with sodium lauryl sulfate as the surfactant, acetic acid as the buffer, and sodium sulfate as the electrolyte. It was initiated at room temperature and pH 4 with a sodium formaldehyde sulfoxylate, tert-butylhydroperoxide, and iron (ii) sulfate redox system.

Comparative polymer C3 was prepared according to the procedure described in Example 1 of WO 2014/204937.

Comparative polymers C4 and C5 are available from KRATON.

### Example 2

### Particle Size Characterization of Exemplary and Comparative Polymers

Exemplary and comparative polymers as prepared in Example 1 were evaluated for particle size as shown in Table 2.

TABLE 2
Particle Size Characterization

| Sample | Particle Size (nm) |
|---|---|
| P3 | 105 |
| P4 | 314 |
| P5 | 137 |
| P6 | 133 |
| P7 | 342 |
| C3 | 130 |

The particle size distributions of exemplary and comparative polymer was determined by light diffraction using a Malvern Mastersizer 2000 Analyzer equipped with a 2000uP module. Approximately 0.5 g of polymer emulsion samples were pre-diluted into 5 mL of 0.2 weight % active Triton 405 in degassed, DI water (diluents). The pre-diluted sample was added drop-wise to the diluent filled 2000uP module while the module was pumped at 1100 rpm. Red light obscurations were targeted to be between 4 and 8%. Samples were analyzed using a Mie scattering module (particle real refractive index of 1.48 and absorption of zerp: Diluent real refractive index of 1.330 with absorption of zero). A general purpose (spherical) analysis model with “normal sensitivity” was used to analyze the diffraction patterns and convert them into particle size distributions.

### Example 3

### Molecular Weight Characterization of Exemplary and Comparative Polymers

Exemplary and comparative polymers as prepared in Example 1 were evaluated for molecular weight as shown in Table 3.

TABLE 3
Molecular Weight Characterization

| Sample | Molecular Weight (kDa) |
|---|---|
| P1 | 6760 |
| P2 | 1710 |
| P3 | 1690 |
| P6 | 1190 |
| C1 | 2900 |
| C4 | 244 |
| C5 | 144 |

Sample molecular weight were determined by gel permeation chromatography (GPC) on a PLgel MIXED-A LS or Shodex 807L column set using a solvent mixture of tetrahydrofuran/formic acid (100:5 v/v) as the mobile phase. Sample concentration was prepared at 1 mg/mL. GPC eluent flow rate was 0.5 mL/min. Average molecular weights were obtained using both multi-angle light scattering (MALS) and conventional calibration (with poly(meth methacrylate) standards) methods.

### Example 4

### Spray Drying of Exemplary and Comparative Polymers

Exemplary and comparative polymers as prepared in Example 1 were spray dried according to the following procedure. A two-fluid nozzle atomizer was equipped on a Mobile Minor spray dryer (GEA Process Engineering Inc.). The spray drying experiments were performed under an inert atmosphere of nitrogen. The nitrogen supplied to the atomizer at ambient temperature was set at 1 bar and 50% flow, which is equivalent to 6.0 kg/hour of flow rate. The polymer emulsion was fed into the atomizer at about 30 mL/min using a peristaltic pump (Masterflex L/S). Heated nitrogen was used to evaporate the water. The inlet temperature was set at 120° C., and the outlet temperature was equilibrated at 40-50° C. by fine tuning the emulsion feed rate. The resulting polymer powder was collected in a glass jar attached to the cyclone and subsequently vacuum dried at room temperature to removed residual moisture.

### Example 5

Viscosity of Polyacrylate Oil Gel Prepared from Spray Dried Exemplary and Comparative Polymers

The viscosities of exemplary polyacrylate oil gels formed from exemplary and comparative polymers as prepared in Example 1 and spray dried according to the procedure in Example 4 are shown in Table 4.

TABLE 4
Viscosities of Polyacrylate Oil Gel
from Spray Dried Acrylic Polymer

| | | Polymer | | | |
|---|---|---|---|---|---|
| | | Concentration | Mixing | | |
| | | in Oil | Time | Viscosity | |
| Sample | Oil⁺ | (wt %) | (hr) | (cP) | Clarity |
| P1 | CCT | 4 | 1.5 | 38,000* | Clear |
| P2 | CCT | 4 | 2.0 | 4,840* | Clear |
| P3 | CCT | 4 | 2.0 | 3,290* | Clear |
| P4 | CCT | 4 | 1.0 | 1,500** | Clear |
| | SSO | 4 | 1.0 | — | — |
| P5 | SSO | 1 | 1.0 | 12,200* | Clear |
| | SSO | 4 | 1.0 | >100,000* | Clear |
| P6 | CCT | 4 | 1.0 | 2,030* | Clear |
| | SSO | 4 | 1.0 | >100,000 | Clear |
| P7 | SSO | 4 | 1.0 | 3,910* | Clear |
| C1 | CCT | 4 | 1.5 | Unstable | — |
| C2 | SSO | 4 | 1.0 | Unstable | — |
| C3 | CCT | 4 | 1.0 | Unstable | — |
| C4 | CCT | 4 | 1.5 | — | Translucent |
| | SSO | 4 | 2.0 | — | Opaque |
| C5 | SSO | 4 | 2.0 | 390* | Clear |

⁺CCT = Caprylic/capric triglyceride is available from Rita Corporation; SSO = Sunflower seed oil available from Spectrum.
*Measured with Brookfield viscometer, Spindle S96 at 6 rpm
**Measured with Brookfield viscometer, Spindle S64 at 6 rpm

Exemplary polyacrylate oil gels as evaluated in Table 4 above were formulated by heating the cosmetic oil to 70° C. under stirring (EuroStar 60, IKA) at 500 rpm. Polyacrylate polymer was added into the hot oil under stirring at 70° C. for 1-2 hours. The mixture was then cooled to room temperature.

The results demonstrate that the inventive polyacrylate oil gels exhibit far superior viscosity enhancement and clarity when compared with comparative oil gels prepared from comparative polymers.

### Example 6

Rheology Characterization of Polyacrylate Oil Gels Prepared from Exemplary Polymer and Sunflower Seed Oil

Viscosities of oil gel samples were measured using a Rheometrics RFS III Rheometer with a Couette geometry (bob diameter=32 mm, cup diameter=34 mm, bob length=34 mm). All measurements were performed at a strain of 2%, within the linear viscoelastic regime. All analyses were performed at 20° C., and isothermal frequency sweep was conducted. A logarithmic step ramp method was used ranging over the frequency range of 0.1-100 s⁻¹ with 10 data points per decade after an initial 2 minute equilibration.

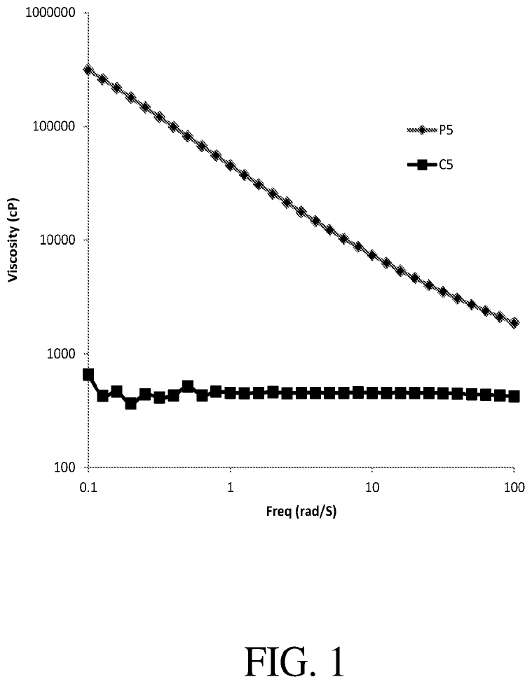

FIG. 1 shows the rheology profiles for 4 weight % exemplary polymer P5 and comparative polymer C5 as prepared in Example 1 above in sunflower seed oil. The exemplary P5 oil gel formed viscous and shear-thinning oil gel with sunflower seed oil, which his highly desirable for leave on skin care formulations, while the comparative C5 oil gel demonstrated a much lower viscosity.

What is claimed is:

1. A polyacrylate oil gel composition comprising:
(a) hydrophobic ester oil; and
(b) one or more polymers comprising polymerized units derived from
(i) 85 to 100 weight % of C₄-C₈ (meth)acrylate monomers, and
(ii) 0 to 15 weight % of high T_{g} monoethylenically unsaturated monomers having a T_{g} of more than 90° C. after polymer formation.

2. The composition of claim 1, wherein the C₄-C₈ (meth)acrylate monomers are selected from the group consisting of ethylhexyl (meth)acrylate, butyl (meth)acrylate, and combinations thereof.

3. The composition of claim 1, wherein one or more polymers comprise polymerized units derived from 50 to 100 weight % butyl (meth)acrylate monomers.

4. The composition of claim 1, wherein the high T_{g} monoethylenically unsaturated monomers are present in an amount of from 0.1 to 10 weight %, based on the total weight of the polymer.

5. The composition of claim 4, wherein the high T_{g} monoethylenically unsaturated monomers comprise one or more of methyl methacrylate, t-butyl methacrylate, styrene, and isobornyl methacrylate.

6. The composition of claim 1, wherein the polymers further comprise 0.01 to less than 0.3 weight % polymerized units derived from crosslinkers.

7. The composition of claim 6, wherein the crosslinkers comprise allyl methacrylate.

8. The composition of claim 1, wherein the hydrophobic ester oil comprises one or more aliphatic C₈-C₂₄ alkyl triglycerides.

9. The composition of claim 1, wherein the polymers have an average particle size of from 50 to 500 nm.

10. A personal care composition comprising a polyacrylate oil gel comprising:
(a) one or more aliphatic C₈-C₂₄ alkyl triglycerides;
(b) one or more polymers comprising polymerized units derived from
(i) 80 to 90 weight % of butyl methacrylate, and
(ii) 10 to 20 weight % of ethylhexyl methacrylate; and
(c) a dermatologically acceptable carrier,
wherein the polymers have an average particle size of from 130 to 140 nm.
